(19) United States
(12) Patent Application Publication
(10) Pub. No.: US 2021/0128848 A1
(43) Pub. Date: May 6, 2021

(54) INHALER MOUTHPIECE DEVICE

(71) Applicant: Prakash MEHTA, Mumbai (IN)

(72) Inventor: Prakash MEHTA, Mumbai (IN)

(21) Appl. No.: 17/128,549

(22) Filed: Dec. 21, 2020

Related U.S. Application Data
(63) Continuation-in-part of application No. 15/151,028, filed on May 10, 2016, now abandoned.

(30) Foreign Application Priority Data
Jul. 15, 2015 (IN) 2676/MUM/2015

(51) Int. Cl. A61M 15/00

(52) U.S. Cl. CPC A61M 15/0025; A61M 15/0065

(57) ABSTRACT

The present invention relates to an inhaler mouthpiece device for use as an accessory aid for metered dose inhalers and spacers used along with such inhalers. The device removably attachable to the metered dose inhaler simplifies and assists a patient's inhaling effort to advance an inhaled medication into lungs of the patient.

### CROSS REFERENCE TO RELATED APPLICATIONS

This patent application claims the benefit of the priority under 35 U.S.C. § 119 b to Indian Patent Application No. 2676/MUM/2015 filed Jul. 15, 2015. This patent application is a Continuation-in-Part of U.S. patent application Ser. No. 15/151,028 filed May 10, 2016. Both of the above patent applications are incorporated by reference herein.

### TECHNICAL FIELD

The present invention relates to a device for use as an accessory aid for metered dose inhalers and spacers used along with such inhalers. The device removably attachable to the metered dose inhaler simplifies and assists a patient's inhaling effort to advance an inhaled medication into lungs of the patient.

### BACKGROUND

Generally, patients suffering from various respiratory diseases, such as Asthma, Chronic Bronchitis, Emphysema and others mostly depend upon various inhalers that include metered dose inhalers (MDIs), dry powder inhalers and other inhalers. Metered dose inhalers are convenient handheld devices and require patients to have good hand-breath coordination so they may simultaneously manage breathing and actuation for effective drug delivery into the lungs. However, it is known that the management and use of the metered dose inhalers pose a significant administration problem as incorrect metered dose inhaler usage technique negatively impacts treatment, compromising on medication delivery, resulting in loss of medication and poor deposition of medication into the lungs. This further adds an economic burden to the patient. The extent of loss of medication is determined on the patient's ability to administer the inhaler usage technique without error and further is determined by the comfort level of the inhaler in the mouth experienced by the user.

In a general administration of the medication by correct metered dose inhaler usage technique, it is required that the patient shakes the inhaler prior to use, exhales fully and away from the inhaler, places the mouthpiece end in the mouth and sealing it with the lips, actuates the inhaler, inhales slowly and deeply, holds breath for up to 10 seconds, and thereafter exhales. Various studies conducted on the correct metered dose inhaler usage techniques and common errors committed by patients while administration of medication with conventional inhalers reveal that 25-30% patients never receive/follow instructions on correct inhaler usage techniques, 39.4% patients inhale fast and not too deep, 41.9% patients fail to hold breath for up to 10 seconds. The conventional inhaler mouthpiece end has an area of 231.0 sq mm which for children and elderly patients, is extremely difficult, uncomfortable, and inconvenient to hold in the mouth while sealing it with the lips and trying to retain their breath up to 10 seconds. Further, in a study conducted by Rice University on “How much medicine makes it to lungs?”, it has been observed that using conventional inhalers with this big mouthpiece, when correct inhaler usage techniques are adopted, it results in 40% of the inhaled medicine reaching the lungs and when various errors are committed while using these inhalers, the results drop from 40% to 7%. This implies that the loss of medication either inside the mouth or backflow through the lips or in any other manner is anywhere from 93% to 60%.

Referring FIGS. 1 and 2, they illustrate a conventional inhaler (100) with a cap (140) in an engaged position and a disengaged position. The inhaler (100) consists of a canister (110) containing medication to be dispensed when pressed at its top end. The canister (110) is inserted into the plastic body/casing (120) of the inhaler (100) from a top end. A mouthpiece end (130) of inhaler (100) is closed by engaging the cap (140). When the cap (140) is disengaged and the inhaler (100) is actuated the medication is dispensed out from the mouthpiece end (130). The limitation of the conventional inhaler (100) is the mouthpiece end (130) has an inconvenient large area. Patients with poor lung condition using the conventional inhaler (100) with inconvenient large area mouthpiece end (130) while attempting to follow correct metered dose inhaler usage technique by holding their breath, experience lack of air and desperately need to take in a breath, for which involuntarily and instantly unseal their lips from the inhaler and open their mouth by terminating the inhaled breath with medication, to relieve them out of this shortness of breath situation. This unsealing of the lips expels any remaining medication inside their throat and mouth which exists the mouth as a back flow. Thus, the medication administered with conventional large mouthpiece end (130) metered dose inhalers is not uniquely directed towards the lungs, and inherently loss of medication takes place mostly in the mouth of the patient as the medication spreads out in the mouth and less dosage is administered into the respiratory stream. Also, in the conventional inhalers due to the structure of the mouthpiece end (130), it has been observed that, over a period of administration, a layer of medication is formed on inner surfaces of the mouthpiece end (130).

Therefore, the predominance of errors found in inhaler usage techniques and the inherent problems associated with the larger mouthpiece end inhalers, highlights the need and importance of alternative devices to mitigate the aforementioned problems associated with conventional inhalers.

### SUMMARY

This summary is provided to introduce concepts related to an inhaler mouthpiece device. This summary is neither intended to identify essential features of the present invention nor is it intended for use in determining or limiting the scope of the present invention.

Accordingly, an aspect of the present invention discloses an inhaler mouthpiece device comprising a cap removably fitted to a metered dose inhaler or a spacer used with the metered dose inhaler; and a hollow mouthpiece attached to the cap to form a single piece inhaler mouthpiece device, said hollow mouthpiece has a diameter of 5 mm to 10 mm to be well fitted into mouth of a user and extends out 20 mm to 30 mm from the cap to place in the mouth of user. The hollow mouthpiece is sealed by lips of the user, in a course of using the inhaler and holding the inhaled breath by the user, for delivering medicine directly to the back of mouth or throat of the user, with breathing flow of air into the lungs of the user, with less loss of medication in the mouth of the user and without any loss of medication due to unsealing of lips in the course of holding breath by the user.

According to an embodiment, the hollow mouthpiece preferably extends out 23 mm to 28 mm from the cap to place in the mouth of the user.

According to an exemplary embodiment, the cap is snap fitted to a metered dose inhaler and with modifications as required to fit the outlet end of the spacers. The inhaler mouthpiece device has a plug of suitable shape and size to smooth fit into the hollow mouthpiece at an inhalation end, to act as a closure to protect the mouthpiece from getting dirty and unclean. The shape of this plug is such that it can easily be pulled out before use and pushed in after use.

According to the embodiment, the hollow mouthpiece has one of a circular, elliptical, oblong, rectangular, or square cross-section.

According to another aspect, the present invention discloses a metered dose inhaler, consisting of an inhaler body; a cap, either removably fitted to the inhaler or integrally molded as part of the inhaler body; a hollow mouthpiece attached to the cap to form a single piece inhaler mouthpiece device, said hollow mouthpiece has a smaller diameter of 5 mm to 10 mm to be well fitted into mouth of a user and extends out 20 mm to 30 mm from the cap to place in the mouth of user, wherein the hollow mouthpiece is sealed by lips of the user, in a course of using the inhaler and holding the inhaled breath by the user, for delivering medicine directly to the back of mouth or throat of the user, with breathing flow of air into the lungs of the user, with less loss of medication in the mouth of the user and without any loss of medication due to unsealing of lips in the course of holding breath by the user.

### BRIEF DESCRIPTION OF ACCOMPANYING DRAWINGS

The above and other aspects, features, and advantages of certain exemplary embodiments of the present invention will be more apparent from the following description taken in conjunction with the accompanying drawings in which:

FIG. 1 illustrates a side view of a conventional inhaler with a cap in an engaged position;

FIG. 2 illustrates the side view of the conventional inhaler with the cap in a disengaged position;

FIG. 3 illustrates a side view of an inhaler mouthpiece device, according to an aspect of the present invention;

FIG. 4A illustrates different perspective view of the inhaler mouthpiece device, according to an embodiment of the present invention;

FIG. 4B illustrates different perspective view of the inhaler mouthpiece device, according to an embodiment of the present invention;

FIG. 5 illustrates a side view of an inhaler with the inhaler mouthpiece device in a disengaged position, according to the embodiment of the present invention;

FIG. 6 illustrates a side view of the inhaler with the inhaler mouthpiece device in an engaged position, according to the embodiment of the present invention;

FIG. 7 illustrates a side sectional view of the inhaler with the inhaler mouthpiece device integrally moulded to the inhaler, according to other embodiment of the present invention; and

FIG. 8 illustrates a side view of a spacer, one end of which is attached to the inhaler and the other to the inhaler mouthpiece, according to still another embodiment of the present invention.

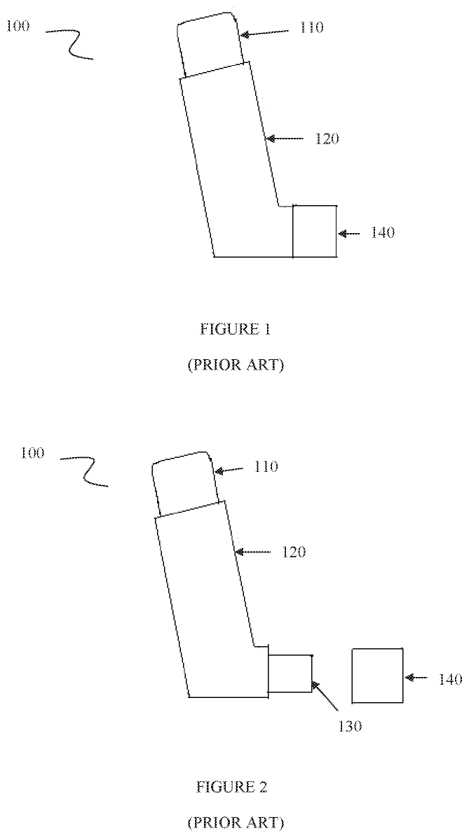

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure.

Throughout the drawings, it should be noted that like reference numbers are used to depict the same or similar elements, features, and structures.

### DETAILED DESCRIPTION

In the following description, for the purpose of explanation, specific details are set forth in order to provide an understanding of the present invention. It will be apparent, however, to one skilled in the art that the present invention may be practiced without these details. One skilled in the art will recognize that embodiments of the present invention, some of which are described below, may be incorporated into several applications.

In general, the present invention claims an inhaler mouthpiece device removably fitted to a mouthpiece end of a metered dose inhaler. The inhaler mouthpiece device simplifies and assists a patient's inhaling effort to advance an inhaled medication into lungs of the patient. The inhaler mouthpiece device has a cap and a hollow mouthpiece is attached to the cap to form a single piece inhaler mouthpiece device. The hollow mouthpiece has a smaller diameter that comfortably fits into mouth of a user and extends from the cap to place in the mouth of user such that in a course of correct metered dose inhaler usage technique of holding breath for up to 10 seconds, there is no inconvenience for the user and the medication is administered directly to back of mouth or throat of the user, with breathing flow of air into the lungs of the user, with less loss of medication in the mouth of the user and without any loss of medication due to unsealing of lips or opening of the mouth in the course of holding breath. The extending length of the hollow mouthpiece from the cap into the mouth of the user addresses the inherent loss of medication posed by the conventional inhalers, wherein the medication greatly spreads out in the mouth before reaching into the lungs. The extending length of the hollow mouthpiece ensures that a greater portion of medication directed towards back of the mouth or throat of the user, advances into the lungs of the user before spreading out in the mouth of the user, thus the inhaler mouthpiece device simplifies and assists the user's inhaling effort to advance an inhaled medication into lungs and eliminates any backflow medication loss. Also, the structure of the inhaler mouthpiece device ensures that no layers of medication is formed on inner surfaces of the inhaler.

Other aspects, advantages, and salient features of the invention will become apparent to those skilled in the art from the following detailed description, which, taken in conjunction with the annexed drawings, discloses exemplary embodiments of the invention.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents. All the terms and expressions in the description are only for the purpose of the understanding and nowhere limit the invention.

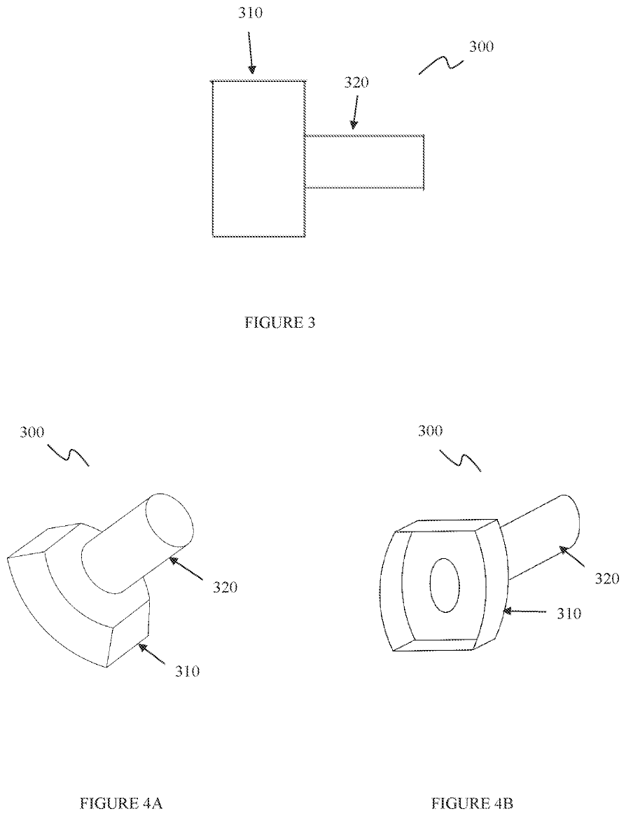

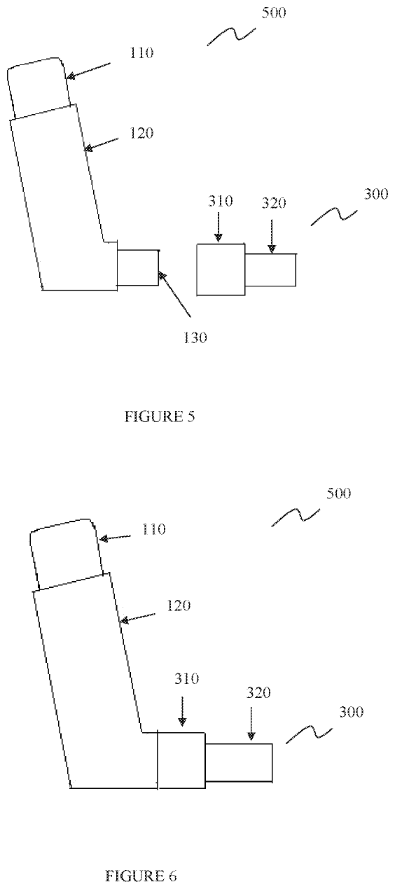

Referring FIGS. 3-8, discloses an inhaler mouthpiece device (300), a cap (310), a hollow mouthpiece (320), an inhaler (500, 700) with the inhaler mouthpiece device (300) and an inhaler (800) used with a spacer (150) and the inhaler mouthpiece device (300), an inlet end (160) and an outlet end (170) of the spacer (150).

Referring FIGS. 3-8, discloses an inhaler mouthpiece device (300) comprising a cap (310) removably fitted to a metered dose inhaler (500, 700) or a spacer (150) used with the metered dose inhaler (800); and a hollow mouthpiece (320) attached to the cap (310) to form a single piece inhaler mouthpiece device (300), said hollow mouthpiece (320) has a smaller diameter of 5 mm to 10 mm to be well fitted into mouth of a user and extends out 20 mm to 30 mm from the cap (310) to place in the mouth of user. The hollow mouthpiece (320) is sealed by lips of the user, in a course of holding breath by the user, for delivering medicine directly to the back of mouth or throat of the user, with breathing flow of air into the lungs of the user, with less loss of medication in the mouth of the user and without any loss of medication due to unsealing of lips in the course of holding breath by the user.

According to the embodiment, the hollow mouthpiece (320) is fixed and part of the cap (310). The inhaler mouthpiece device (300) is removably attached to a mouthpiece end (130) of the inhaler (500) or moulded as part of the body of the inhaler (700) in any shape, form or size as part of the inhaler body. As the inhaler mouthpiece device (300) is integrally moulded, the user may simply shake the inhaler before use and, and use directly, with no need to remove the cap. This also enables use of the metered dose inhaler without removing the mouthpiece device from it before use for speed and convenience. The inhaler mouthpiece device (300) is fitted to the mouthpiece end, and in this state the inhaler can be used without removing the cap. The inhaler mouthpiece device (300) in an exemplary embodiment is snap fitted to the mouthpiece end. The inhaler mouthpiece device (300) may be of varying sizes according to the type of the inhaler. The inhaler mouthpiece device (300) in an exemplary embodiment has a plug (not shown) of suitable shape and size to smooth fit into the hollow mouthpiece at an inhalation end, to act as a closure to protect the mouthpiece (320) from getting dirty and unclean. The shape of this plug is such that it may easily be pulled out before use and pushed in after use.

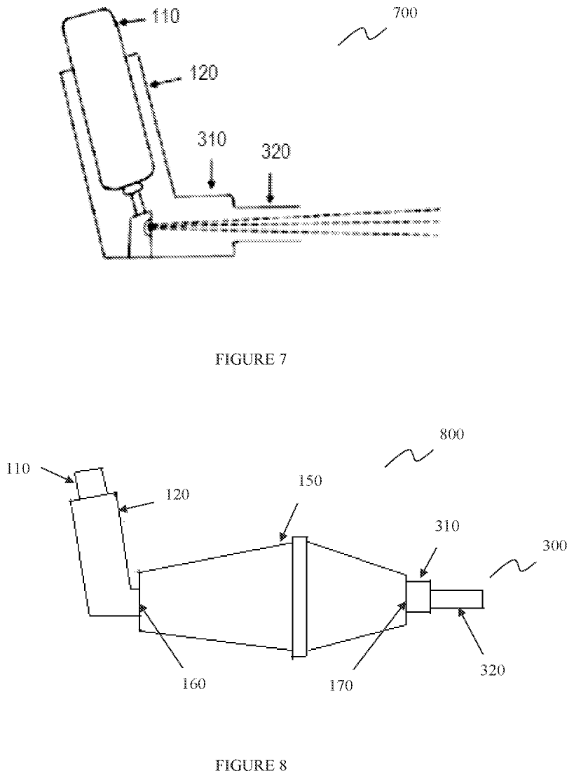

Referring FIG. 7 illustrates sectional view of the inhaler mouthpiece device (300), wherein the cap (310) is integrally moulded to the body (120) of the inhaler (700). The flow stream of the medication from the bottom of the canister (110) is dispensed after actuating the inhaler at top, through a nozzle at the bottom and through the hollow mouthpiece (320). The cap (310) moulded may be of any suitable shape and size.

Referring FIG. 8, the inhaler mouthpiece device (300) of required size is removably attached to the spacer (150) at an outlet end (170) and an inlet end of the spacer (150) is connected to the mouthpiece end (130) of the inhaler (800). The medication exits the mouthpiece end (130) of the inhaler (800) into the spacer (150) through inlet end (160) and comes out of the outlet end (170) into the inhaler mouthpiece device (300) when inhaled by the user.

According to the present invention, the cap (310), the hollow mouthpiece may have same or different material that includes but not limited to plastic and other materials.

According to an exemplary embodiment, the inhaler mouthpiece device (300) is snap fitted to the inhaler (500, 700, 800).

The hollow mouthpiece (320) of the inhaler mouthpiece device (300) is easy and comfortable for the user to place in the mouth. The hollow mouthpiece (320) may be any shape and not limited to elliptical, square, oblong, rectangle. The cap (310) with hollow mouthpiece (320) of inhaler mouthpiece device (300) is integrally moulded to the inhaler body (120).

The extending hollow mouthpiece (320) enables the user to hold his breath comfortably for a longer period compared to the bigger mouthpiece for correct inhaler usage technique. With the inhaler mouthpiece device (300) easy coordination of hand and the breathing in (inhaling) while pressing the inhaler to release the dose is made possible. The medication is directed efficiently to the back of mouth or throat of the user, so that it easily and rapidly enters the air flow into the lungs, with more of the medication. The user/patient has to just suck in the dose with minimal effort, with less or no strain. Thus, the inhaler mouthpiece device (300) ensures less loss of medication in the mouth and eliminates back flow dues to unsealing of lips, simplifying the usage for young children and elderly patients. The simple, convenient and easy to use inhaler mouthpiece device (300) provides greater satisfaction, better results of medication administration into the lungs of the user.

The inconvenient conventional metered dose inhaler mouthpiece end (130) has a large area of 231 sq mm, a length of 14 mm, a width of 21 mm and an average height of 11 mm, which is to be placed into the mouth for administration of the medication. However, it has been observed that out of the 14 mm length of the mouthpiece, and since everyone's mouth is different, and therefore taking average sizes, nearly 9 mm of this length is taken up by the lips placed around it and sealing it during use. Thus, the mouthpiece end (130) with less than or equal to 5 mm (ie. 14 mm-9 mm) extension into the mouth results in the loss of the medication in the mouth, as greater amount of the medication primarily spreads out in the mouth before reaching to the lungs of the user. Whereas, the hollow mouthpiece (320) of the inhaler mouthpiece device (300) according to the present invention, has length 20 mm to 30 mm extending from the cap (310), a diameter 5 mm (equivalent to an area of 19.50 sq mm) to 10 mm (equivalent to an area 78.0 sq mm). Therefore, the area of the hollow mouthpiece (320) of the inhaler mouthpiece device (300) is 8.3%-33.1% of that of conventional inhaler mouthpiece, which is critical. The hollow mouthpiece (320) has an additional length of 6 mm to 16 mm when compared to the conventional inhaler mouthpiece length of 14 mm, and after considering the length taken up by the lips sealing the hollow mouthpiece (320) by 9 mm it has a length of 11 mm to 21 mm extending into the mouth when compared to the conventional inhaler mouthpiece. The additional extending length of the hollow mouthpiece (320) is critical to ensure that there is less loss of medication in the mouth and greater amount of medication administered advances directly to back of mouth or throat of the user with the inhalation into the lungs.

According to the present invention, any lengths, below or above this effective net lengths of 11 mm to 21 mm, indicate unexpected results of greater and more retention and loss of medication in the mouth. Therefore, it is determined that the size and length of the mouthpiece is a crucial factor in the deposition and loss of medication in the mouth. This is represented by an exemplary Table 1, wherein the inventor has represented criticality with respect to the dimensions of the inhaler mouthpiece device, specifically to the lengths and diameters of the hollow mouthpiece (320). The test and experiments conducted by the inventor are as follows:

Example 1: In the experiment, the parameters of the hollow mouthpiece (320) of the present invention considered for experiment are length and diameter. An effective length of the mouthpiece for the study is based on the lengths from the lips abutting a cap end and extending into the mouth and not any length remaining outside the lips (if any). Length of 10 mm closest to 11 mm is considered as starting length, the length of the conventional metered dose inhalers being 14 mm, for experiment and then 2-3 mm incremental lengths are considered for determining criticality. The experiments are performed for a given diameter with variable lengths. The diameters considered are of 5 mm, 10 mm and 15 mm and for each of these diameters, lengths of 10, 13, 15, 18, 20, 23, 25, 28, 30, 33, 35, 38, 40, 43 and 45 mm are considered.

By varying the parameters, the quantity of medication deposited/lost in the mouth is observed as a satisfaction level. These satisfaction levels are given the labels A, B and C, where A=Above Average, indicating lesser losses, B=Average, indicating average losses, and C=Below Average, indicating more losses, in the mouth. Satisfaction level A indicates least deposition and Satisfaction level C indicates highest deposition. The Satisfaction level attributes of A, B and C are further quantified on a scale of 1 to 10, scale 1 representing lowest loss of medication in the mouth and scale 10 representing highest loss of medication in the mouth. Accordingly, in the table given below, scale between 1 and 3 is represented by a satisfaction level A, scale between 4 and 7 is represented by a satisfaction level B and scale between 8 to 10 is represented by a satisfaction level C.

Given below is a table representing the observed and experienced results of the experiment conducted:

TABLE 1

| | D | | | | | |
|---|---|---|---|---|---|---|
| L mm | 5 mm | | 10 mm | | 15 mm | |
| 10 | C | 9 | C | 9 | C | 9 |
| 13 | C | 8 | C | 8 | C | 8 |
| 15 | B | 7 | B | 7 | B | 7 |
| 18 | B | 6 | B | 6 | C | 7 |
| 20 | A | 3 | A | 3 | B | 6 |
| 23 | A | 2 | A | 2 | B | 6 |
| 25 | A | 2 | A | 2 | B | 6 |
| 28 | A | 2 | A | 2 | B | 6 |
| 30 | A | 3 | A | 3 | B | 6 |
| 33 | B | 6 | B | 6 | B | 7 |
| 35 | B | 6 | B | 6 | B | 7 |
| 38 | B | 6 | B | 6 | C | 7 |
| 40 | C | 7 | C | 7 | C | 8 |
| 43 | C | 8 | C | 8 | C | 8 |
| 45 | C | 8 | C | 8 | C | 9 |

The tabulated results signify criticality indicating that for a hollow mouthpiece (320) diameter of 5 mm-10 mm, the critical lengths of 20 mm-30 mm and preferably, 23 mm-28 mm provide the best results showing less loss of medication in the mouth. The length parameters of 15 mm and 35 mm, show a greatly increased effect on the amount of deposition/loss of medication in the mouth which is three times more than that for a length of 20 mm-30 mm. This table accordingly reflects the criticality of the length of the mouthpiece. The criticality of the diameter is also likewise reflected in the table as the diameter 5 mm-10 mm is more comfortable in the mouth for better sealing of the lips, thereby lowering the loss in the mouth by the sealing of the lips over the mouthpiece.

Example 2: The inventor to further establish loss of medication in the mouth has conducted a second experiment. While using a steroid medication inhaler, as there will be disbursement in the mouth and throat of the user of some of this medication, it needs to be rinsed out otherwise it may cause a fungal infection known as Thrush (Candidiasis). The exemplary experiment is conducted using a conventional inhaler with the bigger mouthpiece and with the inhaler mouthpiece device (300) attached to the inhaler (500, 700, 800) of the present invention. A clear transparent container is utilized to store the rinsed out steroid medication using both these types of mouthpieces. It has been observed, that after rinsing the mouth with the exact same quantity of water, that in case of conventional inhaler, in each rinsing of the medication a dense cloudy thick liquid is visible to the naked eye in the container. The visible precipitate clearly indicates that the medication is spread out in the mouth, resulting in greater disposition/loss of medication in the mouth. Whereas, with the inhaler mouthpiece device (300) of the present invention a far less dense precipitate is visible in the rinsed water in the container. This establishes that there is less loss of medication in the mouth with the use of the inhaler mouthpiece device (300) when compared to the conventional inhaler mouthpiece.

There have been described and illustrated herein several embodiments of an inhaler mouthpiece device. While particular embodiments of the invention have been described, it is not intended that the invention be limited thereto, as it is intended that the invention be as broad in scope as the art will allow and that the specification be read likewise. The type of fit, materials, shape of the elements are disclosed, the embodiments are not limited to the described herein above, many modifications with the scope of the invention are possible which are evident to the person skilled in the art. It is understood that the above description is intended to be illustrative, and not restrictive.

I claim:

1. An inhaler mouthpiece device comprising:
a cap removably fitted to a metered dose inhaler or a spacer used with the metered dose inhaler; and
a hollow mouthpiece attached to the cap to form a single piece inhaler mouthpiece device, said hollow mouthpiece has a diameter of 5 mm to 10 mm to be well fitted into mouth of a user and extends out 20 mm to 30 mm from the cap to place in the mouth of the user, wherein the hollow mouthpiece is sealed by lips of the user, in a course of holding breath by the user, for delivering medicine directly to the back of mouth or throat of the user, with breathing flow of air into the lungs of the user, with less loss of medication in the mouth of the user and without any loss of medication due to unsealing of lips in the course of holding breath by the user.

2. The device as claimed in claim 1, wherein the hollow mouthpiece extends out 23 mm to 28 mm from the cap to place in the mouth of the user.

3. The device as claimed in claim 1, wherein the hollow mouthpiece has one of a circular, elliptical, oblong, rectangular, or square cross-section.

4. A metered dose inhaler, comprising:
an inhaler body;
a cap, either removably fitted to the inhaler or integrally molded as part of the inhaler body; and
a hollow mouthpiece attached to the cap to form a single piece inhaler mouthpiece device, said hollow mouthpiece has a diameter of 5 mm to 10 mm to be well fitted into mouth of a user and extends out 20 mm to 30 mm from the cap to place in the mouth of the user, wherein the hollow mouthpiece is sealed by lips of the user, in the course of holding breath by the user, for delivering medicine directly to the back of mouth or throat of the user, with breathing flow of air into the lungs of the user, with less loss of medication in the mouth of the user and without any loss of medication due to unsealing of lips in the course of holding breath by the user.
